(19) United States
(12) Patent Application Publication
(10) Pub. No.: US 2010/0151018 A1
(43) Pub. Date: Jun. 17, 2010

(54) SUSTAINED-RELEASE LEVETIRACETAM COMPOSITION AND PREPARATION PROCESS

(75) Inventors: Hervé Brochart, Wittelsheim (FR); Estelle Foucher, Kienheim (FR)

Correspondence Address: BROWDY AND NEIMARK, P.L.L.C.;624 NINTH STREET, NW, SUITE 300, WASHINGTON, DC, 20001-5303, US

(73) Assignee: RD- Pharmagal, illkirch Graffenstaden (FR)

(21) Appl. No.: 12/525,920

(22) PCT Filed: Feb. 5, 2008
(86) PCT No.: PCT/FR2008/000130

§ 371 Date: Mar. 1, 2010

(30) Foreign Application Priority Data
Feb. 5, 2007 (FR) 0700790

(51) Int. Cl. A61K 9/36; A61K 31/4015; A61P 25/08; B05D 1/02

(52) U.S. Cl. 424/465; 514/424; 427/2.15; 427/2.14

(57) ABSTRACT

A subject of the present invention is a novel formulation of levetiracetam making it possible to obtain a solid pharmaceutical composition, particularly intended for oral administration, for the sustained release of levetiracetam. A subject of the invention is also a process for the preparation of such a pharmaceutical composition.

The present invention belongs to the field of pharmacy, particularly the field of galenic pharmacy. A subject of the invention is a novel formulation of levetiracetam making it possible to obtain a sustained-release solid pharmaceutical composition particularly intended for oral administration.

Levetiracetam is a pyrrolidone derivative (the S-enantiomer of a-ethyl-2-oxo-1-pyrrolidine acetamide), chemically unrelated to existing anti-epileptic active ingredients.

Levetiracetam is indicated in the epileptic patient in association with the treatment of partial crises. It is currently available in the form of immediate-release tablets marketed by UCB Pharma under the trade name Keppra®. These tablets contain a dose of 250 mg or 500 mg, as well as 750 mg in the USA. The initial treatment for an adult is 500 mg twice daily, knowing that this dose can be increased to 1500 mg twice daily depending on the clinical response and tolerance to the treatment.

The absorption of the levetiracetam is rapid after oral administration of these tablets: the maximum plasma concentrations (Cmax) are reached 1.3 hours after administration and the bioavailability is approximately 100%. In adults the plasma half-life is short, 7 hours+/−1, and varies neither with the dose, nor with the repetition of the doses. Given the excellent bioavailability of levetiracetam and its rapid elimination, it was clearly apparent that a formulation making it possible to sustain the release of the active ingredient would have the advantage of reducing the daily administrations.

The consequences would be an increase in compliance with treatment for the epileptic patient as well as a reduction in the side effects conventionally linked with the plasmatic peak.

The advantages of the sustained release formulations are well known to a person skilled in the art and such possibilities have already been studied for levetiracetam. However the major difficulty in the development of such a formulation resides in the very good solubility of levetiracetam in water (approximately 104.0 g/100 ml) combined with the need to have a minimum of 500 mg of active ingredient available per administration unit.

It is therefore clear to a person skilled in the art that given the dose of levetiracetam necessary for a therapeutic activity, only a small quantity of excipient can be added without increasing the size of the tablet such that it becomes difficult to swallow. Now, it is also known to a person skilled in the art that an active ingredient which is highly soluble in water requires a significant quantity of excipients if a sufficiently sustained release is to be achieved.

Thus the international application WO01/51033 describes a solid pharmaceutical composition for controlled release which may contain levetiracetam and requires both the presence of a matrix excipient, an enterosoluble polymer and an alkalinizing agent for sustaining the release profile. The examples mentioned correspond to dosages of 120 mg or 240 mg of active ingredient for a tablet with a total mass of 550 mg, i.e. tablets containing a dose of only 22% or 44% weight/weight of active ingredient. These dosages are less than the normal therapeutic dose usually prescribed to an adult.

The International application WO 03/101428 describes a solid pharmaceutical composition which may contain levetiracetam, and is obtained by hot compaction of copolymers of methacrylic acid acting as excipients delaying the release of the active ingredient.

The relatively large proportion of active ingredient (a maximum of 77% weight/weight active ingredient) incorporated in the compositions obtained is however due only to the low solubility in water of the molecule mentioned in the examples (oxcarbazepine: 308 mg/l)

The international application WO 2006/080029 describes sustained-release tablets of levetiracetam, compounds with a core compulsorily comprising, besides levetiracetam, a high-viscosity hydrodispersible polymer controlling the release of the active ingredient and an optional coating the role of which is also to delay the dispersion of the levetiracetam.

These tablets can contain 40 to 80%, preferentially 50 to 75% by weight of the composition, or also 30 to 85% by weight of the levetiracetam core.

However, the total mass of the tablets is very high, of the order of 850 mg for tablets containing a 500 mg dose of levetiracetam, and 1150 mg for tablets with 750 mg of active ingredient, due, in particular, to the presence in the core of the high-viscosity water-soluble polymer controlling the release of the active ingredient. This makes their ingestion difficult.

Finally, the international application WO 06/088864 describes a levetiracetam-based composition releasing the active ingredient bimodally, one part being released immediately and the other in a modified manner.

It therefore appears clearly that the state of the art teaches that the very high solubility of levetiracetam in water, combined with the need to have a minimum of 500 mg of the latter available per administration unit oblige a person skilled in the art to use large quantities of excipients sustaining the release of the levetiracetam and as a result make it difficult, or even impossible, for the patient to take the tablet.

A real need therefore exists for a formulation of levetiracetam allowing the production of pharmaceutical compositions for sustained release of the active ingredient comprising a large quantity of said active ingredient and a small quantity of excipients.

By “sustained release”, is meant that the active ingredient present in the solid pharmaceutical composition, is released from said composition in a low dose, continuously, over a long period of time and not suddenly.

By “high-viscosity water-soluble polymer” is meant a water-soluble polymer which confers upon the solution in which it is dissolved at a concentration of 2% (weight/weight), a viscosity at 25° C. greater than 20 cps.

By “low-viscosity water-soluble polymer” is meant a water-soluble polymer which confers upon the solution in which it is dissolved at a concentration of 2% (weight/weight) a viscosity at 25° C. greater than or equal to 20 cps, (measured using a Brookfield viscosimeter in a sample volume of 500 ml).

One of the objectives of the present invention is to provide a solid pharmaceutical composition with a high dose of levetiracetam, exhibiting sustained release of said active ingredient and comprising only a small amount of excipient.

This aim is achieved in the composition according to the invention.

The pharmaceutical composition according to the invention can comprise up to 96% by weight of active ingredient. The properties of said pharmaceutical composition allow ease of ingestion by the patient and therefore good compliance of the latter with the treatment.

Furthermore, the release profile of the active ingredient has moreover the advantage of being independent of the pH, which, in vivo, ensures its release throughout the whole length of the gastro-intestinal tract.

This composition is characterized particularly by the absence from its core of any high-viscosity water-soluble compound which exerts an influence on the release of the levetiracetam. It is therefore understood that the control of the release of the active ingredient (levetiracetam) is obtained with the composition according to the invention only by the very particular composition of the coating.

Thus, a subject of the present invention is a solid pharmaceutical composition which can comprise at least
a core, strictly free of any high-viscosity water-soluble compound, comprising levetiracetam in a quantity comprised between 75 and 96%, preferentially 80 and 96%, very preferentially 85 and 96% by weight with respect to the total weight of the pharmaceutical composition and at least one lubricant; and
a coating which is semi-permeable to water.

According to a particular aspect of the invention, the core can also comprise at least one low-viscosity water-soluble polymer.

According to the invention, said solid pharmaceutical composition can take all forms compatible with its function, particularly with the fact that it has to be ingested. Advantageously, said solid pharmaceutical composition can take the form of a tablet.

According to the invention, the core can comprise 80 to 99%, preferentially 85 to 99%, very preferentially 90 to 99%, by weight of levetiracetam with respect to the weight of said core.

According to the invention, the levetiracetam can be present in the pharmaceutical composition in a minimum quantity of 200 mg, preferentially at least 350 mg, very preferentially at least 500 mg. Advantageously, said pharmaceutical composition can comprise levetiracetam in a maximum quantity comprised between 500 and 1000 mg.

According to the invention, the low-viscosity water-soluble polymer can be introduced into the pharmaceutical composition via a solution for wetting the levetiracetam used during the process for the preparation of said composition. Said wetting solution can be chosen from the solutions of this type well known to a person skilled in the art. Said solution can be composed of at least a low-viscosity water-soluble polymer and a solvent comprising water or a mixture of solvents comprising water.

Said low-viscosity water-soluble polymer can be chosen from polyvinylpyrolidone (or Povidone), hydroxypropylmethyl cellulose (of low viscosity), hydroxypropyl cellulose, carboxymethyl cellulose or its sodium salt. Preferentially, the excipient is polyvinylpyrrolidone or hydroxypropylmethyl cellulose (of low viscosity). A preferred low-viscosity water-soluble polymer is Povidone K90. Of course the low-viscosity water-soluble polymer can be constituted by a mixture of at least two low-viscosity water-soluble polymers chosen from those described previously. Said low-viscosity water-soluble polymer (or said mixture of low-viscosity water-soluble polymers) can be in a quantity comprised between 1 and 15%, preferentially between 1 and 10%, by weight with respect to the weight of said core.

According to the invention, the solvent of the wetting solution can be chosen from water, ethanol, isopropanol, or a mixture of said solvents. Preferentially, said solvent is water or also a water-ethanol or water-isopropanol mixture.

According to the invention, the lubricant can be chosen from sodium stearyl fumarate, magnesium stearate, stearic acid, talc, hydrogenated castor oil, glycerol behenate or also polyethylene glycol or sodium benzoate, preferentially magnesium stearate or sodium stearyl fumarate. Said lubricant can be in a quantity comprised between 0.2 and 10%, preferentially between 0.5 and 5%, by weight with respect to the weight of said core.

According to a variant of the invention, the core can moreover comprise a flow-improving agent such as for example colloidal silica.

Said flow-improving agent can be in a quantity comprised between 0.05 and 3%, preferentially between 0.1 and 1%, by weight with respect to the weight of said core.

Advantageously, the coating can be a film-forming coating, still more advantageously a semi-permeable film-forming coating.

Said coating can allow control of the release of the active ingredient from the pharmaceutical composition, such that 2 hours after ingestion no more than 20%, preferentially no more than 15%, of the levetiracetam is released, at 4 hours, no more than 20 to 45%, preferentially no more than 15 to 40%, of the levetiracetam is released, at 8 hours, no more than 45 to 85%, preferentially no more than 40 to 80%, of the levetiracetam are released, and at 16 hours no less than 85%, preferentially no less than 80%, of the levetiracetam is released.

According to the invention, the coating can be obtained by the use of at least one water-insoluble polymer.

According to a variant of the invention, the coating which is semi-permeable to water can be obtained by the combination of at least one polymer which is insoluble in water and at least one polymer which is soluble in water (water-soluble), of high or low viscosity.

According to the invention, the water-insoluble polymer can be chosen from ethyl cellulose or polyvinyl alcohol. Preferentially the water-insoluble polymer is ethyl cellulose. Advantageously, the water-insoluble polymer can be present in said composition in a quantity which can represent 20% to 99%, preferentially 30% to 90%, still more preferentially between 40% and 80% by weight of the dry coating.

According to the invention, the water-soluble polymer can be chosen from polyvinylpyrrolidone, polyethylene glycol, hydroxypropylmethyl cellulose, hydroxypropyl cellulose, carboxymethyl cellulose, its sodium salt, or also xanthan gum. Preferentially the water-soluble polymer can be chosen from polyvinylpyrrolidone, polyethylene glycol, hydroxypropylmethyl cellulose. Advantageously, the water-soluble polymer can be present in the coating in a quantity which can represent 5% to 45%, preferentially 10% to 40%, still more preferentially 15% to 35%, by weight of the dry coating.

According to the invention, the ratio of the quantity of water-insoluble polymer to the quantity of water-soluble polymer can be determined in order to obtain the desired release profile of the levetiracetam. Thus, the ratio of the quantity of water-insoluble polymer to the quantity of water-soluble polymer can be comprised between 0.8 and 10, preferentially between 1 and 6, still more preferentially between 2 and 5.

Advantageously and irrespective of the variant according to the invention, the coating can optionally comprise at least one plasticizer, with the purpose of conferring a certain elasticity on said coating. Said plasticizer can be chosen from those described in the “Handbook of Pharmaceutical Excipients, Kibbe A. H., Pharmaceutical Press, London, (2000). Among the latter, by way of example mention may be made of polyethylene glycol, propylene glycol, dibutyl phthalate, triethyl citrate or also dibutyl sebacate. Preferentially the chosen plasticizer is of water-soluble type such as polyethylene glycol and propylene glycol. Advantageously, said plasticizer can be present in the composition in a quantity which can represent 2% to 40%, preferentially 5% to 25% by weight of the dry coating.

According to a variant of the invention, the polyethylene glycol can simultaneously act as a water-soluble polymer and a plasticizing agent, in which case it must be present in a quantity comprised between 5% and 50% preferentially between 10 and 45% by weight of the dry coating.

According to the invention, the coating can represent 1 to 20%, preferentially 1 to 10% of the total weight of the pharmaceutical composition.

A subject of the invention is also a process for the preparation of a pharmaceutical composition according to the invention in which
in a first stage (stage 1) said core comprising levetiracetam, at least one solution intended for wetting the levetiracetam, and at least one lubricant are prepared; and
in a second stage (stage 2) the core obtained in stage 1 is coated.

According to the invention stage 1 of preparation of the core can be carried out by implementing the following sub-stages:
in a first sub-stage 1A levetiracetam is prepared in the form of granules;
in a second sub-stage 1B the granules obtained in the first sub-stage 1A are mixed with at least one lubricant and optionally with a flow-improving agent;
in a third sub-stage 10 the mixture obtained in sub-stage 1B is compressed in order to obtain the core of said pharmaceutical composition.

Said first sub-stage 1A of preparation of levetiracetam granules can be carried out by wet granulation, or by granulation in a fluidized bed or also by compaction, preferentially by granulation in a fluidized bed.

According to a variant of the invention, if the granulation is carried out by wet granulation, the latter can be carried out in a standard or a high-speed mixer.

According to another variant of the invention, if the granulation is carried out by granulation in a fluidized bed the latter can be carried out in a system such as those manufactured by Glatt or also by Niro.

Said sub-stage 1A can be carried out by introducing the levetiracetam available in powder form, previously passed through a sizing grid, for example with a mesh opening comprised between 0.7 and 0.9 mm, preferentially 0.8 mm, into a fluidized-bed system such as those mentioned previously. The granulation is accomplished by spraying a wetting solution comprising a solvent and optionally, depending on the desired variant of the product, at least one low-viscosity water-soluble polymer.

The product obtained is then dried and sized, for example on an oscillating granulator of Frewitt (Switzerland) or Erweka (Germany) type in order to obtain the desired granules of levetiracetam.

According to the invention, sub-stage 1B can be carried out by mixing the granules of levetiracetam obtained in sub-stage 1A, with a lubricant as described previously using a mixer which is conventionally used for this type of operation and known to a person skilled in the art.

The mixture obtained by this present technique, without it being necessary to incorporate into the latter other excipients known to a person skilled in the art, with granulometry, density as well as flow characteristics making them easily compressible.

According to the invention, sub-stage 10 can be carried out by compressing the product obtained in sub-stage 1B, for example using a Fette P2 type press (Germany) in order to obtain the cores of the pharmaceutical composition according to the invention.

According to the invention, the tablets can take all the desired shapes such as for example a circular shape with a diameter comprised between 10 and 15 mm, preferentially with a diameter of 12R12, or also an oblong shape the length of which can be comprised between 12 and 22 mm, preferentially 17 mm and the width comprised between 4 and 10 mm, preferentially 8.5 mm.

According to the invention, stage 2 of coating the core can be carried out by implementing the following sub-stages:
in a first sub-stage 2A at least one water-insoluble polymer and optionally at least one water-soluble polymer of high or low viscosity, and optionally a plasticizer, are dissolved in a mostly non-aqueous solvent such as denatured alcohol, ethanol or also isopropanol, in order to obtain a coating solution;
in a second sub-stage 2B the coating solution obtained in stage 2A can be sprayed onto the cores obtained in sub-stage 1C of stage 1, in a fluidized-bed granulator or also in a standard or perforated coater.

According to the invention, the nature, quantities and optionally the ratios between the different polymers used for the coating are those described previously.

A subject of the invention is also the use of the process as described previously in the preparation of a pharmaceutical composition for sustained release of the active ingredient, for example of levetiracetam according to the invention.

A subject of the invention is also the use of a pharmaceutical composition according to the invention in the treatment of epilepsy

Other features and advantages of the invention will become apparent on reading the following embodiment examples of the invention.

### EXAMPLE 1

### Levetiracetam Tablet with a Diameter of 12R12 and Hardness Close to 120 Newtons

The following core is produced:

| | | |
|---|---|---|
| Levetiracetam | 500.00 | mg |
| Povidone K90 | 15.00 | mg |
| Demineralized water | 285.00 | mg |
| Sodium stearyl fumarate | 5.00 | mg |
| Total (solvent not included): | 520.00 | mg |

The Povidone K90 is dissolved in the water. The levetiracetam previously passed through a 0.8 mm sizing grid is granulated in a Glatt GPCG-1 granulator in a fluidized bed with the following parameters:

Inlet temperature: 55° C.

Product temperature: 37° C.

Flow rate of air: 70 m3/h

Spraying pressure: 2.2 bars

Nozzle: 1.2 mm

After drying and sizing, for example using an oscillating granulator from Frewitt (Switzerland) or Erweka (Germany), the granulate is then mixed with lubricant (Sodium stearyl fumarate) using a Turbula mixer (WAB Bachoffen, Switzerland) and compressed using a Fette P2 type press (Germany) into tablets of diameter 12R12 with a hardness close to 120 Newtons. The cores are then coated according to the following formula:

| | | |
|---|---|---|
| Core | 520.00 | mg |
| Ethocel STD 100 Premium ® | 25.00 | mg |
| Povidone K90 | 6.50 | mg |
| Polyethylene glycol 1500 | 7.00 | mg |
| Denatured alcohol | 400.00 | mg |
| Total (solvent not included): | 558.50 | mg |

The PEG, Povidone and ethyl cellulose are dissolved in the denatured alcohol. The coating solution is then sprayed onto the cores in a Glatt GPCG-1 type fluidized bed device using the following parameters:

Inlet temperature: 40° C.

Product temperature: 39° C.

Air flow rate: 190 m3/h

Spraying pressure: 2.5 bars

Nozzle: 1.2 mm

### EXAMPLE 2

### Measurements of the Dissolution Profile

The dissolution profile of the coated tablets obtained to Example 1 is measured.

The measurement of the dissolution profile is carried out in a type I (basket) device according to US PHARMACOPOEIA 25 (2000), monograph <711> the stirring device of which rotates at 100 rpm at a temperature of 37° C. The tests are carried out according to the following conditions:

Dissolution medium 0.1 N HCl or 0.05M phosphate buffer pH=7.5

Volume: 1000 ml

Dissolution tool: basket

Dissolution speed: 100 rpm

The following results are obtained

| | 2 h | 4 h | 8 h | 10 h | 16 h | 20 h | 24 h |
|---|---|---|---|---|---|---|---|
| 0.1N HCl | 8% | 27% | 67% | 79% | 97% | 100% | 100% |
| pH = 7.5 | 8% | 25% | 67% | 80% | 98% | 101% | — |

These results show that the release profiles obtained over 24 hours remain independent of the pH:

### EXAMPLE 3

### Oblong Levetiracetam Tablet (17×8.5 mm)

The following core is produced:

| | | |
|---|---|---|
| Levetiracetam | 610.00 | mg |
| Povidone K90 | 18.30 | mg |
| Demineralized water | 345.00 | mg |
| Sodium stearyl fumarate | 5.70 | mg |
| Total (solvent not included): | 634.00 | mg |

The procedure used is the same as for Example 1.

After granulation, drying and sizing, the granulate is then mixed with lubricant (sodium stearyl fumarate) using a Turbula mixer and compressed into oblong tablets 17×8.5 mm using a Fette P2 press.

The cores are then coated according to the following formula:

| | | |
|---|---|---|
| Core | 634.00 | mg |
| Ethocel STD 100 Premium ® | 25.00 | mg |
| Povidone K90 | 6.00 | mg |
| Polyethylene glycol 1500 | 7.00 | mg |
| Denatured alcohol | 400.00 | mg |
| Total (solvent not included): | 672.00 | mg |

The coating parameters are identical to those used for Example 1.

Measurement of the dissolution profile of the coated tablets obtained is carried out as in Example 2.

The following results are obtained

| | 2 h | 4 h | 8 h | 10 h | 16 h | 20 h | 24 h |
|---|---|---|---|---|---|---|---|
| 0.1N HCl | 4% | 27% | 61% | 72% | 90% | 96% | 99% |
| pH = 7.5 | 3% | 27% | 61% | 72% | 88% | 94% | 97% |

The release profiles obtained over 24 hours remain independent of the pH:

### EXAMPLE 4

### Oblong Levetiracetam Tablets (20×8 mm)

The following core is produced:

| | | |
|---|---|---|
| Levetiracetam | 750.00 | mg |
| Povidone K90 | 45.00 | mg |
| Demineralized water | 855.00 | mg |
| Sodium stearyl fumarate | 8.00 | mg |
| Total (solvent not included): | 803.00 | mg |

The procedure is the same as for Example 1.

After granulation, drying and sizing, the granulate is then mixed with lubricant (Sodium stearyl fumarate) using a Turbula mixer and compressed using an Korsch EK/O type alternating press into oblong tablets 20×8 mm.

The cores are then coated according to the following formula:

| | | |
|---|---|---|
| Core | 803.00 | mg |
| Ethocel STD 100 Premium | 37.50 | mg |
| Povidone K90 | 9.75 | mg |
| Polyethylene glycol 1500 | 10.50 | mg |
| Denatured alcohol | 600.00 | mg |
| Total (solvent not included): | 860.75 | mg |

The coating parameters are identical to those used for Example 1.

Measurement of the dissolution profile of the coated tablets obtained is carried out as in Example 2.

The following results are obtained:

| | 2 h | 4 h | 8 h | 10 h | 16 h | 20 h | 24 h |
|---|---|---|---|---|---|---|---|
| 0.1N HCl | 6% | 23% | 64% | 76% | 94% | 100% | — |
| pH = 7.5 | 5% | 23% | 69% | 81% | 96% | 100% | — |

The release profiles obtained over 24 hours remain independent of the pH:

### EXAMPLE 5

### Levetiracetam Tablets with a Diameter of 12R12 with a Hardness Close to 120 Newtons

The following core is produced:

| | | |
|---|---|---|
| Levetiracetam | 500.00 | mg |
| Povidone K90 | 15.00 | mg |
| Demineralized water | 285.00 | mg |
| Sodium stearyl fumarate | 5.00 | mg |
| Total (solvent not included): | 520.00 | mg |

The procedure is the same as for Example 1.

After granulation, drying and sizing, the granulate is then mixed with lubricant using a Turbula mixer and compressed using a Fette P2 type press into tablets with a diameter of 12R12 with a hardness close to 120 Newtons.

The cores are then coated according to the following formulae which differ in their ethyl cellulose/Povidone ratio:

| Formula | A | | B | | C | |
|---|---|---|---|---|---|---|
| Core | 520.00 | mg | 520.00 | mg | 520.00 | mg |
| Ethocel STD 100 Premium ® | 25.00 | mg | 25.00 | mg | 25.00 | mg |
| Povidone K90 | 6.00 | mg | 6.50 | mg | 7.00 | mg |
| Polyethylene glycol 1500 | 7.00 | mg | 7.00 | mg | 7.00 | mg |
| Denatured alcohol | 400.00 | mg | 400.00 | mg | 400.00 | mg |
| Total (solvent not included): | 558.00 | mg | 558.50 | mg | 559.00 | mg |

The coating parameters are identical to those used for Example 1.

Measurement of the dissolution profile of the coated tablets obtained is carried out as in Example 2.

The following results are obtained:

| | 2 h | 4 h | 8 h | 10 h | 16 h | 20 h | 24 h |
|---|---|---|---|---|---|---|---|
| Formula A | 2% | 7% | 37% | 51% | 76% | 85% | 91% |
| Formula B | 8% | 27% | 67% | 79% | 97% | 100% | 100% |
| Formula C | 12% | 37% | 79% | 90% | 101% | — | — |

The greater the quantity of water-soluble polymer the more rapid the release of levetiracetam.

### EXAMPLE 6

### Influence of the Low-Viscosity Water-Soluble Polymer Present in a Composition According to the Invention on the Dissolution

A core (formula A) constituted by 750 mg of pure levetiracetam is produced by direct compression of the active ingredient on a Fette P2 press equipped with oblong punches of 18.5×6.5 mm. This core therefore contains no polymer whatsoever.

The dissolution of this core is compared to that of 750 mg of pure levetiracetam in powder (formula B), as well as to a core produced according to Example 4 (formula C) comprising Povidone as a low-viscosity water-soluble polymer.

The dissolution method is as follows: USP type 1 (basket) apparatus, phosphate buffer pH 6.8, 900 ml, speed of rotation of baskets 100 rpm, measurement after 15 minutes by spectrophotometry at 220 nm.

The results are as follows:

| | Formula | | |
|---|---|---|---|
| | Formula A | Formula B (pure | Formula C (active |
| | (compressed pure | active ingredient | ingredient + |
| | active ingredient) | in powder) | polymer tablets) |
| Dissolved | 99.22 | 98.25 | 98.96 |
| fraction (%) | | | |

These results clearly demonstrate that the low-viscosity water-soluble polymer present in formula C according to the invention exerts no appreciable influence on the dissolution.

1. Solid pharmaceutical composition comprising at least a core, strictly free of any high-viscosity water-soluble compound, comprising levetiracetam in a quantity comprised between 75 and 96% by weight with respect to the total weight of the pharmaceutical composition and at least one lubricant; and a coating which is semi-permeable to water.

2. Pharmaceutical composition according to claim 1, wherein the core also comprises at least one low-viscosity water-soluble polymer.

3. Pharmaceutical composition according to claim 1, wherein said core comprises 80 to 99% by weight of levetiracetam with respect to the weight of said core.

4. Composition according to claim 1, wherein said low-viscosity water-soluble polymer is chosen from polyvinylpyrrolidone (or Povidone), hydroxypropylmethyl cellulose, hydroxypropyl cellulose, carboxymethyl cellulose or its sodium salt.

5. Composition according to claim 1, wherein the low-viscosity water-soluble polymer is in a quantity comprised between 1 and 15% by weight with respect to the weight of said core.

6. Pharmaceutical composition according to claim 1, wherein the lubricant is chosen from sodium stearyl fumarate, magnesium stearate, stearic acid, glycerol behenate or also polyethylene glycol, talc, hydrogenated castor oil, sodium benzoate.

7. Pharmaceutical composition according to claim 1, wherein the lubricant is in a quantity comprised between 0.2 and 10% by weight with respect to the weight of said core.

8. Pharmaceutical composition according to claim 1, further comprising a flow-improving agent.

9. Pharmaceutical composition according to claim 7, wherein said flow-improving agent is in a quantity comprised between 0.05 and 3% by weight with respect to the weight of said core.

10. Pharmaceutical composition according to claim 1, wherein the semi-permeable coating comprises at least one water-insoluble polymer or a combination of at least one water-insoluble polymer and at least one water-soluble polymer.

11. Pharmaceutical composition according to claim 1, wherein the water-insoluble polymer is chosen from ethyl cellulose, polyvinyl alcohol.

12. Pharmaceutical composition according to claim 1, wherein the water-insoluble polymer present in the coating is present in a quantity which can represent between 20% to 99% by weight of the dry coating.

13. Pharmaceutical composition according to claim 1, wherein the water-soluble polymer present in the coating is chosen from polyvinylpyrrolidone, polyethylene glycol, hydroxypropylmethyl cellulose, hydroxypropyl cellulose, carboxymethyl cellulose, its sodium salt, or also xanthan gum, preferentially polyvinylpyrrolidone and hydroxypropylmethyl cellulose.

14. Pharmaceutical composition according to claim 1, wherein the water-soluble polymer present in the coating is present in a quantity which represents 5% to 45% by weight of the dry coating.

15. Pharmaceutical composition according to claim 1, wherein the ratio of the quantity of water-insoluble polymer present in the coating to the quantity of water-soluble polymer present in the coating is comprised between 0.8 and 10.

16. Pharmaceutical composition according to claim 1, wherein the coating further comprises at least one plasticizer.

17. Pharmaceutical composition according to claim 1, wherein the plasticizer is chosen from polyethylene glycol, propylene glycol, dibutyl phthalate, triethyl citrate or also dibutyl sebacate, preferentially polyethylene glycol and the glycol.

18. Pharmaceutical composition according to claim 1, wherein the plasticizer is present in the composition in a quantity which represents 2% to 40% by weight of the dry coating.

19. Pharmaceutical composition according to claim 1, wherein the coating represents 1 to 20% of the total weight of the pharmaceutical composition.

20. Process for the preparation of a pharmaceutical composition as described in claim 1, comprising:
in a first stage (stage 1) preparing said core comprising levetiracetam, at least one solution intended for wetting the levetiracetam, and at least one lubricant; and in a second stage (stage 2) coating the core obtained in stage 1.

21. Process according to claim 20, wherein stage 1 is carried out by implementing the following sub-stages:
in a first sub-stage 1A, preparing levetiracetam in the form of granules;
in a second sub-stage 1B, mixing the granules obtained in the first sub-stage 1A with at least one lubricant and optionally with a flow-improving agent;
in a third sub-stage 1C, compressing the mixture obtained in sub-stage 1B to obtain the core of said pharmaceutical composition.

22. Process according to claim 21, wherein said first sub-stage 1A is carried out by wet granulation, or by granulation in a fluidized bed or also by compaction, preferentially by granulation in a fluidized bed.

23. Process according to claim 21, wherein said first sub-stage 1A is carried out by spraying a wetting solution comprising a solvent and drying and sizing the product obtained.

24. Process according to claim 21, wherein said sub-stage 1B is carried out by mixing the granules of levetiracetam obtained in sub-stage 1A with a lubricant using a mixer.

25. Process according to claim 21, wherein said sub-stage 1C is carried out by compressing the product obtained in sub-stage 1B using a press.

26. Process according to claim 21, wherein stage 2 of coating the core is carried out by implementing the following sub-stages: in a first sub-stage 2A, dissolving at least one water-insoluble polymer and in a non-aqueous solvent, in order to obtain a coating solution; in a second sub-stage 2B, spraying the coating solution obtained in stage 2A onto the cores obtained in sub-stage 1C of stage 1, in a fluidized-bed granulator or in a standard or perforated coater.

27. Preparation of a pharmaceutical composition using the process as described in claim 21 for sustained release of an active ingredient, for example levetiracetam.

28. Preparation of a pharmaceutical composition using the process as described in claim 21, the pharmaceutical composition comprising at least a core, strictly free of any high-viscosity water-soluble compound, comprising levetiracetam in a quantity comprised between 75 and 96% by weight with respect to the total weight of the pharmaceutical composition and at least one lubricant; and a coating which is semi-permeable to water.

29. A method of treating epilepsy using a pharmaceutical composition as described in claim 1.

30. Pharmaceutical composition according to claim 1, wherein the quantity of levetiracetam is between 80 and 96% by weight with respect to the total weight of the pharmaceutical composition and at least one lubricant.

31. Pharmaceutical composition according to claim 1, wherein the quantity of levetiracetam is between 85 and 96% by weight with respect to the total weight of the pharmaceutical composition and at least one lubricant.

32. Pharmaceutical composition according to claim 3, wherein said core comprises 90 to 99% by weight of levetiracetam with respect to the weight of said core.

33. Pharmaceutical composition according to claim 3, wherein said core comprises 95 to 99% by weight of levetiracetam with respect to the weight of said core.

34. Pharmaceutical composition according to claim 4, wherein said low-viscosity water-soluble polymer is chosen from polyvinylpyrrolidone or hydroxypropylmethyl cellulose.

35. Pharmaceutical composition according to claim 5, wherein the low-viscosity water-soluble polymer is in a quantity comprised between 1 and 10% by weight with respect to the weight of said core.

36. Pharmaceutical composition according to claim 6, wherein the lubricant is chosen from magnesium stearate or sodium stearyl fumarate.

37. Pharmaceutical composition according to claim 7, wherein the lubricant is in a quantity comprised between 0.5 and 50% by weight with respect to the weight of said core.

38. Pharmaceutical composition according to claim 8, wherein the flow-improving agent comprises colloidal silica.

39. Pharmaceutical composition according to claim 9, wherein said flow-improving agent is in a quantity comprised between 0.1 and 1% by weight with respect to the weight of said core.

40. Pharmaceutical composition according to claim 11, wherein the water-insoluble polymer is ethyl cellulose.

41. Pharmaceutical composition according to claim 12, wherein the water-insoluble polymer present in the coating is present in a quantity which can represent between 30% to 90% by weight of the dry coating.

42. Pharmaceutical composition according to claim 12, wherein the water-insoluble polymer present in the coating is present in a quantity which can represent between 40% and 80% by weight of the dry coating.

43. Pharmaceutical composition according to claim 13, wherein the water-soluble polymer present in the coating is chosen from polyvinylpyrrolidone and hydroxypropylmethyl cellulose.

44. Pharmaceutical composition according to claim 14, wherein the water-soluble polymer present in the coating is present in a quantity which represents 10% to 40% by weight of the dry coating.

45. Pharmaceutical composition according to claim 14, wherein the water-soluble polymer present in the coating is present in a quantity which represents 15% to 35% by weight of the dry coating.

46. Pharmaceutical composition according to claim 15, wherein the ratio of the quantity of water-insoluble polymer present in the coating to the quantity of water-soluble polymer present in the coating is comprised between 1 and 5.

47. Pharmaceutical composition according to claim 15, wherein the ratio of the quantity of water-insoluble polymer present in the coating to the quantity of water-soluble polymer present in the coating is comprised between 2 and 5.

48. Pharmaceutical composition according to claim 17, wherein the plasticizer is chosen from polyethylene glycol and the glycol.

49. Pharmaceutical composition according to claim 18, wherein the plasticizer is present in the composition in a quantity which represents 5% to 25% by weight of the dry coating.

50. Pharmaceutical composition according to claim 19, wherein the coating represents 1 to 10% of the total weight of the pharmaceutical composition.

51. Pharmaceutical composition according to claim 19, wherein the coating represents 1 to 5% of the total weight of the pharmaceutical composition.

52. Process according to claim 22, wherein said first sub-stage 1A is carried out by granulation in a fluidized bed.

53. Process according to claim 21, wherein in said first sub-stage 1A, depending on the desired variant of the product, the wetting solution further comprises at least one low-viscosity water-soluble polymer.

54. Process according to claim 26, wherein in the first sub-stage 2A, the step of dissolving further comprises dissolving the at least one water-insoluble polymer and at least one high- or low-viscosity water-soluble polymer, in the non-aqueous solvent.

55. Process according to claim 26, wherein in the first sub-stage 2A, the step of dissolving further comprises dissolving the at least one water-insoluble polymer and a plasticizer in the non-aqueous solvent.

56. Process according to claim 26, wherein the non-aqueous solvent is selected from denatured alcohol, ethanol or isopropanol.
